SEQUENCE LISTING (75 sequences)

SEQ ID NO: 1 - DNA, 16 bp, Artificial sequence
  gapmer Nr1
guacttcctattagug

SEQ ID NO: 2 - DNA, 16 bp, Artificial sequence
  gapmer Nr2
gugaagtacttccuau

SEQ ID NO: 3 - DNA, 16 bp, Artificial sequence
  gapmer Nr3
agutggtgaagtacuu

SEQ ID NO: 4 - DNA, 16 bp, Artificial sequence
  gapmer Nr4 aka GM1
cuugcagttggtgaag

SEQ ID NO: 5 - DNA, 16 bp, Artificial sequence
  gapmer Nr5
cactgcttgcagtugg

SEQ ID NO: 6 - DNA, 16 bp, Artificial sequence
  gapmer Nr6
gguaccactgcttgca

SEQ ID NO: 7 - DNA, 16 bp, Artificial sequence
  gapmer Nr7
ccuttggtaccacugc

SEQ ID NO: 8 - DNA, 16 bp, Artificial sequence
  gapmer Nr8
auuttcctttggtacc

SEQ ID NO: 9 - DNA, 16 bp, Artificial sequence
  gapmer Nr9
cacagattttcctuug

SEQ ID NO: 10 - DNA, 16 bp, Artificial sequence
  gapmer Nr10
augactgttgattugc

SEQ ID NO: 11 - DNA, 16 bp, Artificial sequence
  gapmer Nr11
cugatgactgttgauu

SEQ ID NO: 12 - DNA, 16 bp, Artificial sequence
  gapmer Nr12 aka GM2
uagctgatgactguug

SEQ ID NO: 13 - DNA, 16 bp, Artificial sequence
  gapmer Nr13 aka GM3
ucgtagctgatgacug

SEQ ID NO: 14 - DNA, 16 bp, Artificial sequence
  gapmer Nr14
cactcgtagctgauga

SEQ ID NO: 15 - DNA, 16 bp, Artificial sequence
  gapmer Nr15
cagcactcgtagcuga

SEQ ID NO: 16 - DNA, 16 bp, Artificial sequence
  gapmer Nr16
ggacagcactcgtagc

SEQ ID NO: 17 - DNA, 16 bp, Artificial sequence
  gapmer Nr17
ccaggacagcactcgu

SEQ ID NO: 18 - DNA, 16 bp, Artificial sequence
  gapmer Nr18
uauccaggacagcacu

SEQ ID NO: 19 - DNA, 16 bp, Artificial sequence
  gapmer Nr19
uuucatatccaggaca

SEQ ID NO: 20 - DNA, 16 bp, Artificial sequence
  gapmer Nr20
gaccttttcatatcca

SEQ ID NO: 21 - DNA, 16 bp, Artificial sequence
  gapmer Nr21
uagtggtggatctgga

SEQ ID NO: 22 - DNA, 16 bp, Artificial sequence
  gapmer Nr22
uaggatagtggtggau

SEQ ID NO: 23 - DNA, 16 bp, Artificial sequence
  gapmer Nr23
guucacagttacaauc

SEQ ID NO: 24 - DNA, 16 bp, Artificial sequence
  gapmer Nr24
gcacagttcacaguua

SEQ ID NO: 25 - DNA, 16 bp, Artificial sequence
  gapmer Nr25
uggttgcatggtgguc

SEQ ID NO: 26 - DNA, 16 bp, Artificial sequence
  gapmer Nr26
cgutggttgcatggug

SEQ ID NO: 27 - DNA, 16 bp, Artificial sequence
  gapmer Nr27
aucgatgaggtgcacc

SEQ ID NO: 28 - DNA, 16 bp, Artificial sequence
  gapmer Nr28
accttatcgatgaggu

SEQ ID NO: 29 - DNA, 16 bp, Artificial sequence
  gapmer Nr29
agatgaccttatcgau

SEQ ID NO: 30 - DNA, 16 bp, Artificial sequence
  gapmer Nr30
gguggagatgaccuua

SEQ ID NO: 31 - DNA, 16 bp, Artificial sequence
  gapmer Nr31
gugatggtggagauga

SEQ ID NO: 32 - DNA, 16 bp, Artificial sequence
  gapmer Nr32
ugutggtgatggtgga

SEQ ID NO: 33 - DNA, 16 bp, Artificial sequence
  gapmer Nr33
gaugttgttggtgaug

SEQ ID NO: 34 - DNA, 16 bp, Artificial sequence
  gapmer Nr34
ugctggatgttgtugg

SEQ ID NO: 35 - DNA, 16 bp, Artificial sequence
  gapmer Nr35
ugatctgctggatguu

SEQ ID NO: 36 - DNA, 16 bp, Artificial sequence
  gapmer Nr36
cucaatgatctgcugg

SEQ ID NO: 37 - DNA, 16 bp, Artificial sequence
  gapmer Nr37
cgatctcaatgatcug

SEQ ID NO: 38 - DNA, 16 bp, Artificial sequence
  gapmer Nr38
gucctcgatctcaaug

SEQ ID NO: 39 - DNA, 16 bp, Artificial sequence
  gapmer Nr39
aaggtgtcctcgaucu

SEQ ID NO: 40 - DNA, 16 bp, Artificial sequence
  gapmer Nr40
ucucaaaggtgtccuc

SEQ ID NO: 41 - DNA, 16 bp, Artificial sequence
  gapmer Nr41
ucaagcatcgtgtuga

SEQ ID NO: 42 - DNA, 16 bp, Artificial sequence
  gapmer Nr42
ccutcaagcatcgugu

SEQ ID NO: 43 - DNA, 16 bp, Artificial sequence
  gapmer Nr43
uuaccttcaagcaucg

SEQ ID NO: 44 - DNA, 16 bp, Artificial sequence
  gapmer Nr44
ggutcaaagtctcacu

SEQ ID NO: 45 - DNA, 16 bp, Artificial sequence
  gapmer Nr45
uucagcaggtctcuca

SEQ ID NO: 46 - DNA, 16 bp, Artificial sequence
  gapmer Nr46
ggutgttcagcagguc

SEQ ID NO: 47 - DNA, 16 bp, Artificial sequence
  gapmer Nr47
gaugtggttgttcagc

SEQ ID NO: 48 - DNA, 16 bp, Artificial sequence
  gapmer Nr48
caagatgtggttguuc

SEQ ID NO: 49 - DNA, 16 bp, Artificial sequence
  gapmer Nr49
cuucaagatgtgguug

SEQ ID NO: 50 - DNA, 16 bp, Artificial sequence
  gapmer Nr50
ugacttcaagatgugg

SEQ ID NO: 51 - DNA, 16 bp, Artificial sequence
  gapmer Nr51
agctgacttcaagaug

SEQ ID NO: 52 - DNA, 16 bp, Artificial sequence
  gapmer Nr52
cauagctgacttcaag

SEQ ID NO: 53 - DNA, 16 bp, Artificial sequence
  gapmer Nr53
acacatagctgacuuc

SEQ ID NO: 54 - DNA, 16 bp, Artificial sequence
  gapmer Nr54
agcacacatagctgac

SEQ ID NO: 55 - DNA, 16 bp, Artificial sequence
  gapmer Nr55
uucagcacacatagcu

SEQ ID NO: 56 - DNA, 16 bp, Artificial sequence
  gapmer Nr56
gauggcttcagcacac

SEQ ID NO: 57 - DNA, 16 bp, Artificial sequence
  gapmer Nr57
ugatagtgagcatguc

SEQ ID NO: 58 - DNA, 16 bp, Artificial sequence
  gapmer Nr58
uuattggagatgaucg

SEQ ID NO: 59 - DNA, 16 bp, Artificial sequence
  gapmer Nr59
uguctttattggagau

SEQ ID NO: 60 - DNA, 16 bp, Artificial sequence
  gapmer Nr60
ggatgtctttattgga

SEQ ID NO: 61 - DNA, 16 bp, Artificial sequence
  gapmer Nr61
cuaggatgtctttauu

SEQ ID NO: 62 - DNA, 16 bp, Artificial sequence
  gapmer Nr62
aucaatgtagtggauc

SEQ ID NO: 63 - DNA, 16 bp, Artificial sequence
  gapmer Nr63
agctcatcaatgtagu

SEQ ID NO: 64 - DNA, 16 bp, Artificial sequence
  gapmer Nr64
ugagtagctcatcaau

SEQ ID NO: 65 - DNA, 16 bp, Artificial sequence
  gapmer Nr65
auucaaatagtgtcuu

SEQ ID NO: 66 - DNA, 16 bp, Artificial sequence
  gapmer Nr66
cugcagcaccagcugg

SEQ ID NO: 67 - DNA, 16 bp, Artificial sequence
  gapmer Nr67
ccutcaggacatccau

SEQ ID NO: 68 - DNA, 16 bp, Artificial sequence
  gapmer Nr68
aggatttcatcaccaa

SEQ ID NO: 69 - DNA, 16 bp, Artificial sequence
  gapmer Nr69
uaaccaggatttcauc

SEQ ID NO: 70 - DNA, 16 bp, Artificial sequence
  gapmer Nr70
aaugcttcatcctcuc

SEQ ID NO: 71 - DNA, 16 bp, Artificial sequence
  gapmer Nr71
caactcatagcttaua

SEQ ID NO: 72 - DNA, 16 bp, Artificial sequence
  gapmer Nr72
guuttattattacaaa

SEQ ID NO: 73 - DNA, 16 bp, Artificial sequence
  gapmer Nr73
cuutggttttattauu

SEQ ID NO: 74 - DNA, 15 bp, Artificial sequence
  negative control oligonucleotide
aacacgtctatacgc

SEQ ID NO: 75 - DNA, 982 bp, Homo sapiens
cacuaauaggaaguacuucaccaacugcaagcagugguaccaaaggaaaaucuguggcaaaucaacagucaucagcuacgagugcuguccuggauaugaaaaggucccuggggagaagggcuguccagcagcccuaccacucucaaaccuuuacgagacccugggagucguuggauccaccaccacucagcuguacacggaccgcacggagaagcugaggccugagauggaggggcccggcagcuucaccaucuucgccccuagcaacgaggccugggccuccuugccagcugaagugcuggacucccuggucagcaaugucaacauugagcugcucaaugcccuccgcuaccauauggugggcaggcgaguccugacugaugagcugaaacacggcaugacccucaccucuauguaccagaauuccaacauccagauccaccacuauccuaaugggauuguaacugugaacugugcccggcugcugaaagccgaccaccaugcaaccaacgggguggugcaccucaucgauaaggucaucuccaccaucaccaacaacauccagcagaucauugagaucgaggacaccuuugagacccuucgggcugcuguggcugcaucagggcucaacacgaugcuugaagguaacggccaguacacgcuuuuggccccgaccaaugaggccuucgagaagaucccuagugagacuuugaaccguauccugggcgacccagaagcccugagagaccugcugaacaaccacaucuugaagucagcuaugugugcugaagccaucguugcggggcugucuguagagacccuggagggcacgacacuggaggugggcugcagcggggacaugcucacuaucaacgggaaggcgaucaucuccaauaaagacauccuagccaccaacggggugauccacuacauugaugagcuacucaucccagacucagccaagacacuauuugaauuggcu

(19) United States
(12) Patent Application Publication
(10) Pub. No.: US 2024/0141340 A1
(43) Pub. Date: May 2, 2024

(54) ANTISENSE OLIGONUCLEOTIDES FOR USE IN THE TREATMENT OF CORNEAL DYSTROPHIES

(71) Applicant: ProQR Therapeutics II B.V., Leiden (NL)

(72) Inventors: Elisabeth Laurentina Wilhelmina Maria VAN MIERLO, Leiden (NL); Gerardus Johannes PLATENBURG, Leiden (NL); Aliye Seda YILMAZ-ELIS, Leiden (NL)

(21) Appl. No.: 18/279,485

(22) PCT Filed: Mar. 4, 2022
(86) PCT No.: PCT/EP2022/055534

§ 371 Date: Aug. 30, 2023

(30) Foreign Application Priority Data
Mar. 5, 2021 (EP) 21 160 987.0

(51) Int. Cl. C12N 15/113

(52) U.S. Cl. CPC C12N 15/113; C12N 2310/11; C12N 2310/315; C12N 2310/321; C12N 2310/3231; C12N 2310/341

(57) ABSTRACT

The invention relates to antisense oligonucleotides (AON) for use in the prevention, treatment, or amelioration of a corneal dystrophy caused by a (mutated) TGFBI gene. More specifically, the invention relates to gapmers for use in the downregulation of TGFBI mRNA expression and/or TGFBI protein expression, thereby preventing, treating, or ameliorating the TGFBI-related corneal dystrophy. The AONs of the present invention prevent or inhibit the occurrence of corneal deposits due to (mutated) TGFBI genes.

### FIELD OF THE INVENTION

The invention relates to the field of medicine. Moreover, the invention relates to the field of antisense oligonucleotides (AONs), such as gapmers, for use in the treatment, prevention, or amelioration of corneal dystrophies, by downregulating the expression of (mutated) human TGFBI transcript and the encoded TGFBI protein.

### BACKGROUND OF THE INVENTION

Corneal dystrophies are a group of genetic, often progressive, eye disorders in which abnormal material accumulates in the clear (transparent) outer layer of the eye (the cornea). The cornea covers the iris, pupil, and anterior chamber. It consists of five distinct layers from the anterior to the posterior: corneal epithelium, Bowman's layer (or membrane), corneal stroma, Descemet's membrane, and the corneal endothelium. Corneal deposits can accumulate in different layers and in different structures such as filaments, granules or blobs depending on the type of corneal dystrophy. Corneal deposits can cause impaired sight due to an opaque cornea. Other symptoms of corneal dystrophies are eye pain, sensitivity to light and recurrent corneal erosions. Most corneal dystrophies are associated with a mutation in the TGFBI gene. Some examples of TGFBI-related corneal dystrophies are epithelial basement membrane dystrophy (EBMD), Reis Bucklers corneal dystrophy (RBCD), Thiel Behnke corneal dystrophy (TBCD), Lattice Corneal Dystrophy classic (LCD), Granular Corneal Dystrophy classic (GCD1) and granular corneal dystrophy type II or Avellino Corneal Dystrophy (ACD). Over sixty TGFBI mutations have been reported. Depending on the specific mutation, corneal deposits will accumulate as filament, granules, or blobs in a variety of corneal layers.

The TGFBI gene codes for the Transforming Growth Factor Beta-Induced protein (TGFBIp; sometimes referred to as BIGH3, big-h3, βigh3, βigh3, or keratoepithelin (KE)), a 68-kDa extracellular matrix (ECM) protein composed of 683 amino acids. TGFBIp is known to exist ubiquitously in various organs including heart, liver, pancreas, skin, bone, tendon, endometrium, kidney, and blood plasma. The role of TGFBIp is not completely understood. The protein is thought to play pivotal roles in physiological and pathogenic responses by mediating cell adhesion, migration, proliferation, and differentiation. TGFBIp is also associated with progression, dissemination, metastasis, and suppression of malignant tumors. In the cornea, TGFBIp is secreted in the ECM and thought to play a role in the maintenance of the ECM and in corneal wound healing. It is expressed mainly in the corneal epithelium, in association with collagen type VI in the stroma, at the stroma/Descemet membrane interface in Fuch's Endothelial Corneal Dystrophy (FECD), and in retrocorneal fibrous membranes. About 60% of corneal TGFBIp is found covalently linked to collagen type XII. However, its precise role in the eye is largely unknown. The way different mutations in the TFGBI gene cause different phenotypic corneal dystrophies also remains unclear. It is believed that the processing of mutant TGFBIp causes the formation of peptide seeds, which co-assemble with other proteins in the cornea and form deposits. It has been shown that the protein composition of corneal deposits differs between GCD1 and LCD1 aggregates. Mutation-specific differences in processed TGFBIp were also found, which are thought to be the root cause of variable phenotypes in TGFBI-related corneal dystrophies. (Courtney et al. 2015. Invest Ophthalmol Vis Sci 56(8):4653-4661). Knockdown of the TGFBI gene in the cornea could inhibit the formation of insoluble aggregates/corneal deposits in all TGFBI-related corneal dystrophies. Mouse studies have shown that TGFBI^{-/-} mice are viable and live a normal lifespan. Their vision is normal, and no structural changes of the cornea were detected (Poulsen et al. 2018. FEBS J 285(1):101-114). Allele-specific gene silencing of TGFBI mRNA by a siRNA in a cell line transiently expressing TGFBI-Arg124Cys, which causes LCD1, resulted in a decrease of aggregate formation (Courtney et al. 2014. Invest Ophthalmol Vis Sci 55(2):977-985). The object of the present invention is to provide an alternative approach to prevent and/or treat TGFBI-related genetic eye diseases that affect the cornea.

### SUMMARY OF THE INVENTION

The present invention relates to an antisense oligonucleotide (AON) capable of downregulating the expression of human Transforming Growth Factor Beta-Induced (TGFBI) transcript in a target cell, wherein the AON is 90% to 100% complementary to a consecutive stretch of nucleotides within the human TGFBI mRNA sequence of SEQ ID NO:75, wherein the AON consists of 16 to 30 linked nucleotides, and wherein the AON comprises (i) a gap segment consisting of at least ten deoxynucleotides, (ii) a 5′ wing segment consisting of at least three linked nucleotides, and (iii) a 3′ wing segment consisting of at least three linked nucleotides, wherein the gap segment is positioned between the 5′ and 3′ wing segments, and wherein each wing segment comprises at least one nucleotide with a non-naturally occurring chemical modification in the sugar moiety.

In one embodiment, the AON according to the invention is for use in the prevention, treatment, or amelioration of a corneal dystrophy, preferably a TGFBI-related corneal dystrophy, such as epithelial basement membrane dystrophy (EBMD), Reis Bucklers corneal dystrophy (RBCD), Thiel Behnke corneal dystrophy (TBCD), Lattice Corneal Dystrophy classic (LCD), Granular Corneal Dystrophy classic (GCD1), granular corneal dystrophy type II (GCD2), or Avellino Corneal Dystrophy (ACD). In one embodiment, the corneal dystrophy is caused by a mutated TGFBI gene, or the transcript thereof encoding the TGFBI protein. In a preferred embodiment, the 5′ and/or the 3′ wing segment consists of LNA nucleotides. In another preferred embodiment, at least one wing segment comprises a nucleotide with a sugar moiety that is mono- or di-substituted at the 2′, 3′ and/or 5′ position, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O—, S-, or N-alkyl; —O—, S-, or N-alkenyl; —O—, S-, or N-alkynyl; —O—, S-, or N-allyl; —O-alkyl-O-alkyl; -methoxy; -aminopropoxy; -methoxyethoxy; -dimethylaminooxyethoxy; and -dimethylaminoethoxyethoxy. In another preferred embodiment, the AON comprises at least one phosphorothioate (PS) internucleoside linkage, preferably wherein all internucleoside linkages in the AON are modified with PS. In yet another preferred embodiment, the 3′ and/or 5′ wing segment comprises or consists of three nucleotides and the gap segment comprises or consists of ten nucleotides, as exemplified in the accompanying examples. In a further preferred embodiment, the AON is 100% complementary to a consecutive stretch of nucleotides within the sequence of SEQ ID NO:75. Preferably, the AON according to the invention comprises or consists of a sequence selected from the group consisting of SEQ ID NO:1, 2, 3, 4, 5, 6, 7, 8, 9, 10, 11, 12, 13, 14, 15, 16, 19, 25, 26, 27, 30, 31, 33, 36, 37, 40, 41, 42, 43, 44, 46, 47, 48, 49, 50, 51, 52, 53, 54, 55, 56, 57, 58, 59, 60, 61, 62, 64, 65, 67, 68, 70, 71, 72, and 73, preferably from the group consisting of SEQ ID NO:1, 2, 3, 4, 5, 6, 7, 8, 9, 10, 11, 12, 13, 14, 15, 27, 33, 40, 42, 46, 47, 48, 49, 50, 51, 52, 53, 54, 55, 56, 57, 58, 59, 60, 62, 65, 70, 71, 72, and 73. Highly preferred is an AON with a sequence selected from the group consisting of SEQ ID NO:4, 12, and 13.

The present invention also relates to a pharmaceutical composition comprising an AON according to the invention and a pharmaceutically acceptable carrier. The invention further relates to a method for treating a TGFBI-related corneal dystrophy in a human subject in need thereof, comprising the step of administering to the subject an AON according to the invention, or a pharmaceutical composition according to the invention, wherein the administration is by intravitreal injection or topical application. Preferably, the administration is by topical application to reach the epithelium and more posterior layers of the cornea, wherein the AON of the present invention is presented to the cornea in a pharmaceutical composition that allows the entry of the AON into the cornea, such as for example by using a viscosifying polymer. The viscosifying polymer is preferably hydroxypropyl methylcellulose (HPMC). In yet another aspect, the invention relates to a use of an AON according to the invention for use in the manufacture of a medicament for the treatment, prevention, or amelioration of a TGFBI-related corneal dystrophy.

### BRIEF DESCRIPTION OF THE DRAWINGS

FIG. 1 shows the reduction of TGFBI mRNA expression in human A549 cells after transfection with three gapmers: GM1, GM2, and GM3. % KD relates to the percentage knock-down. NT means non-transfected and Ctr(−) is a transfection with a negative control gapmer.

FIG. 2 shows the reduction of TGFBI mRNA expression in human A549 cells after 48 h of gymnotic uptake (no transfection reagents) of three gapmers: GM1, GM2, and GM3. % KD relates to the percentage knock-down. NT means non-transfected and Ctr(−) is a treatment with a negative control gapmer.

FIG. 3 shows the reduction of TGFBI mRNA expression in human A549 cells after 72 h of gymnotic uptake (no transfection reagents) of two gapmers: GM2 and GM3, in an experiment that also relates to protein expression reduction (see FIG. 4). % KD relates to the percentage knock-down. NT means non-transfected and Ctr(−) is a treatment with a negative control gapmer.

FIG. 4 shows the reduction of TGFBI protein expression in human A549 cells after 72 h of gymnotic uptake (no transfection reagents) of two gapmers: GM2 and GM3, in an experiment that also relates to protein expression reduction (see FIG. 3). % KD relates to the percentage knock-down. NT means non-transfected and Ctr(−) is a treatment with a negative control gapmer.

FIG. 5 shows the reduction in TGFBI mRNA expression in human primary keratocytes after transfection with gapmers GM1, GM2, and GM3 in comparison to a mock transfection and a transfection with a negative control gapmer.

### DETAILED DESCRIPTION OF THE INVENTION

There is a clear need to have therapeutic compounds for the prevention, treatment, or cure of TGFBI-related corneal dystrophies. Numerous mutations in the TGFBI gene have been identified and that have been associated with corneal dystrophies. Examples are p.Arg124Leu for Reis-Bücklers corneal dystrophy (RBCD), p.Arg555Gln for Thiel-Behnke corneal dystrophy (TBCD), p.Arg124Cys for lattice corneal dystrophy type 1 (LCD1), p.Arg555Trp for granular corneal dystrophy type 1 (GCD1), and p.Arg124His for granular corneal dystrophy type 2 (GCD2). Based on the finding that using siRNA it appeared possible to decrease aggregate formation (see above), the inventors reasoned that by using alternative means a more robust and alternative approach may be possible to downregulate the TGFBI expression and thereby diminish or inhibit the appearance of corneal deposits, and through this, prevent, cure, or treat TGFBI-related corneal dystrophies. The inventors used an approach that has been applied in other circumstances, namely the targeted downregulation of mRNA expression by applying antisense oligonucleotides (AONs), to target the TGFBI mRNA and through the hybridization of the AON to its target mRNA sequence, bring about degradation of the target molecule. The inventors of the present invention envisioned that such may be feasible by applying certain methods and means that were proven to be functional in the eye in the treatment of other inherited eye diseases. For instance, for Leber's Congenital Amaurosis type 10 (LCA10), a phase 2/3 clinical study is currently ongoing with an antisense oligonucleotide (AON) with the INN sepofarsen that can cause the skip of an aberrant 128 bp exon present in the CEP290 pre-mRNA harbouring the c.2991+1655A>G mutation (WO 2012/168435; WO 2013/036105; WO 2016/135334). Similar exon skipping efforts are currently ongoing for different types of Usher syndrome (WO 2016/005514; WO 2017/186739; WO 2018/055134). However, in the current case of TGFBI downregulation, such would not entail splice modulation. Rather, the inventors of the present invention envisioned using AONs to target the mRNA encoding TGFBIp and to lower its expression, predominantly through nuclease-mediated breakdown by using a so-called ‘gapmer’ which is a stretch of DNA nucleotides flanked on either end with a stretch of RNA nucleotides, further harbouring (potentially) a variety of chemical modifications. The hybridization of the gapmer to the TGFBI transcript would result in a double stranded complex that is prone to be degraded by nucleases. An example of gapmer-mediated downregulation is the mutant allele-specific targeting of rhodopsin P23H (pre-)mRNA in the treatment of autosomal dominant Retinitis Pigmentosa (adRP) for which clinical studies have also been initiated (WO 2016/138353). Gapmers have been used in pre-clinical studies using breast and lung cancer models for instance by targeting MALAT1. These studies have resulted in an anti-tumor and anti-metastatic outcome (WO 2013/096837; Arun et al. Therapeutic targeting of long non-coding RNAs in cancer. Trends Mol Med. 2018. 24:257-277; Gutschner et al. The noncoding RNA MALAT1 is a critical regulator of the metastasis phenotype of lung cancer cells. Cancer Res. 2013. 73:1180-1189).

The inventors of the present invention hypothesized that by using a gapmer it could potentially be possible to target the TGFBI transcript in corneal cells, subsequently have the cellular machinery in the corneal target cell breakdown the double stranded complex that is formed and thereby diminish the TGFBI-dependent deposits, with an ultimate result of treating the corneal dystrophy. For this, they initially designed 73 gapmers by a so-called gapmer macro walk (see the accompanying examples). From these experiments, several gapmers that gave significant downregulation of TGFBI expression, such GM1, GM2, and GM3, were used for further studies.

The present invention relates to an antisense oligonucleotide (AON) capable of downregulating the expression of human Transforming Growth Factor Beta-Induced (TGFBI) transcript in a target cell, wherein the AON is 90% to 100% complementary to a consecutive stretch of nucleotides within the human TGFBI mRNA sequence of SEQ ID NO:75, wherein the AON consists of 16 to 30 linked nucleotides, and wherein the AON comprises: (i) a gap segment consisting of at least ten deoxynucleotides, (ii) a 5′ wing segment consisting of at least three linked nucleotides, and (iii) a 3′ wing segment consisting of at least three linked nucleotides; wherein the gap segment is positioned between the 5′ and 3′ wing segments, and wherein each wing segment comprises at least one nucleotide with a non-naturally occurring chemical modification in the sugar moiety. The invention also relates to an antisense oligonucleotide (AON) capable of downregulating the expression of human Transforming Growth Factor Beta-Induced (TGFBI) transcript in a target cell, wherein the AON is at least 90% complementary to a consecutive stretch of nucleotides within the human TGFBI mRNA sequence of SEQ ID NO:75, wherein the AON comprises a 5′ and a 3′ wing segment and a gap segment positioned between the 5′ and 3′ wing segments, wherein each wing segment comprises at least one nucleotide with a non-naturally occurring chemical modification, and wherein the gap segment consists of DNA nucleotides. In one embodiment, the AON of the present invention is for use in the prevention, treatment, or amelioration of a corneal dystrophy, preferably a corneal dystrophy caused by a (mutated) TGFBI gene. In a preferred aspect, the 5′ and/or the 3′ wing segment comprises one or more LNA nucleotides. In a more preferred aspect, the 5′ and 3′ wing segments consist of LNA nucleotides. In another preferred aspect, the present invention relates to a gapmer that comprises or consists of a sequence selected from the group consisting of SEQ ID NO:1, 2, 3, 4, 5, 6, 7, 8, 9, 10, 11, 12, 13, 14, 15, 16, 19, 25, 26, 27, 30, 31, 33, 36, 37, 40, 41, 42, 43, 44, 46, 47, 48, 49, 50, 51, 52, 53, 54, 55, 56, 57, 58, 59, 60, 61, 62, 64, 65, 67, 68, 70, 71, 72, and 73, preferably from the group consisting of SEQ ID NO:1, 2, 3, 4, 5, 6, 7, 8, 9, 10, 11, 12, 13, 14, 15, 27, 33, 40, 42, 46, 47, 48, 49, 50, 51, 52, 53, 54, 55, 56, 57, 58, 59, 60, 62, 65, 70, 71, 72, and 73, and more preferably from the group consisting of SEQ ID NO:4, 12, and 13.

In another preferred aspect, at least one wing segment comprises a nucleotide with a sugar moiety that is mono- or di-substituted at the 2′, 3′ and/or 5′ position, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O—, S-, or N-alkyl; —O—, S-, or N-alkenyl; —O—, S-, or N-alkynyl; —O—, S-, or N-allyl; —O-alkyl-O-alkyl; -methoxy; -aminopropoxy; -methoxyethoxy; -dimethylaminooxyethoxy; and -dimethylaminoethoxyethoxy. Other preferred modifications of the sugar moiety in an AON of the present invention are morpholino modifications, such as phosphorodiamidate morpholino moieties, or a 4′ to 2′ bicyclic sugar moiety, such as a 4′-CH₂—O-2′ or 4′-CH(CH₃)—O-2′ bicyclic sugar moiety. In another preferred embodiment, the AON comprises at least one phosphorothioate (PS) internucleosidic-linkage. More preferably, all internucleosidic linkages in said AON carry a PS modification. Other preferred internucleoside-linkage modifications are outlined elsewhere herein. In yet another preferred aspect, each wing segment of the AON consists of 3 nucleotides and the gap segment consists of 10 nucleotides. Other preferred combinations that be used according to the present invention are 3-9-3, 3-8-3, 4-10-4, 3-10-4, 4-10-3, 4-9-3, 3-9-4, 3-8-4, and 4-8-3 wing-gap-wing segments. The skilled person understands that each wing as well as the gap can be lengthened, shortened to a certain extent to obtain the highest efficiency. All sorts of length modifications are within the present invention if the combination is sufficient in providing an efficient TGFBI downregulation in corneal cells. In any case, each wing can be 2, 3, 4, 5, 6, or 7 nucleotides in length, whereas the gap can be 5, 6, 7, 8, 9, 10, 11, 12, 13, or 14 nucleotides in length. In one preferred aspect of the invention, the AON is 100% complementary to a consecutive sequence within SEQ ID NO:75. In another preferred embodiment, the AON according to the present invention comprises or consists of a sequence selected from the group consisting of SEQ ID NO:1, 2, 3, 4, 5, 6, 7, 8, 9, 10, 11, 12, 13, 14, 15, 16, 19, 25, 26, 27, 30, 31, 33, 36, 37, 40, 41, 42, 43, 44, 46, 47, 48, 49, 50, 51, 52, 53, 54, 55, 56, 57, 58, 59, 60, 61, 62, 64, 65, 67, 68, 70, 71, 72, and 73, preferably from the group consisting of SEQ ID NO:1, 2, 3, 4, 5, 6, 7, 8, 9, 10, 11, 12, 13, 14, 15, 27, 33, 40, 42, 46, 47, 48, 49, 50, 51, 52, 53, 54, 55, 56, 57, 58, 59, 60, 62, 65, 70, 71, 72, and 73, and more preferably from the group consisting of SEQ ID NO:4, 12, and 13.

The present invention further relates to a pharmaceutical composition comprising an AON according to the present invention, and a pharmaceutically acceptable carrier. In a preferred aspect, the AON of the present invention is delivered to the cornea through a topical administration, which means that the pharmaceutical composition is applied directly on the eye itself. In a preferred embodiment, the pharmaceutical composition comprises, besides the AON and a solvent, also a viscosifying polymer to allow the AON to enter the corneal layers. Preferably, the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine. Most preferred is HPMC.

In yet another preferred aspect, the invention relates to a method of treating a TGFBI-related corneal dystrophy in a human subject in need thereof, comprising the step of administering to the subject an AON that is capable of downregulating the expression of human TGFBI transcript, wherein the AON is 90% to 100% complementary to a consecutive stretch of nucleotides within the human TGFBI mRNA sequence of SEQ ID NO:75, wherein the AON consists of 16 to 30 linked nucleotides, and wherein the AON comprises: (i) a gap segment consisting of at least ten deoxynucleotides, (ii) a 5′ wing segment consisting of at least three linked nucleotides, and (iii) a 3′ wing segment consisting of at least three linked nucleotides; wherein the gap segment is positioned between the 5′ and 3′ wing segments, and wherein each wing segment comprises at least one nucleotide with a non-naturally occurring chemical modification in the sugar moiety. In yet another preferred aspect, the invention relates to a method of treating a TGFBI-related corneal dystrophy in a human subject in need thereof, comprising the step of administering to the subject a pharmaceutical composition according to the invention, wherein the administration is by intravitreal injection or by topical application. The administration is preferably by topical administration using in the pharmaceutical composition a viscosifying polymer as outlined above, preferably HPMC (also referred to as ‘hypromellose’), which will assist in the penetration of the AON into the corneal layers where it should execute its targeting of the TGFBI transcript. The method of the present invention enables the targeting of corneal cells after topical administration and allows the entry of the AON of the present invention to enter the cells that suffer from a TGFBI mutation causing the corneal dystrophy. WO 2021/018750 (herein incorporated in its entirety) discloses methods and means for corneal delivery of nucleic acid molecules such as AONs. When the administration is through direct intravitreal injection, it is preferred that the AON is delivered ‘naked’, without the aid of a carrier, but such is not explicitly excluded.

In yet another aspect, the invention relates to a use of an AON according to the invention in the manufacture of a medicament for the treatment, prevention, or amelioration of TGFBI-related corneal dystrophies.

Herein, the use of the singular includes the plural unless specifically stated otherwise. As used herein, the use of “or” means “and/or” unless stated otherwise. The use of “including” as well as other forms, such as “includes” and “included” is not limiting. Terms such as “element” or “component” encompass both elements and component that comprise more than one subunit, unless specifically stated otherwise. All documents, or portions of documents, cited herein, are hereby expressly incorporated by reference for the portions of the documents discussed herein, as well as in their entirety.

It is understood that the sequence set forth in each SEQ ID NO in the examples contained herein is independent of any modification to the sugar moiety, an internucleoside linkage, or a nucleobase. As such, antisense compounds defined by a SEQ ID NO may comprise, independently, one or more modifications to a sugar moiety, an internucleoside linkage, or a nucleobase.

Unless otherwise indicated, the following terms have the following meaning:

“2′-O-methoxyethyl” (also 2′-MOE, 2′-methoxyethoxy, or 2′-O(CH₂)₂—OCH₃) refers to an O-methoxy-ethyl modification at the 2′ position of a sugar ring, e.g., a furanose ring. A 2′-O-methoxyethyl modified sugar is a modified sugar.

“2′-MOE nucleoside” (also 2′-O-methoxyethyl nucleoside, or 2′-methoxyethoxy nucleoside) means a nucleoside comprising a 2′-MOE modified sugar moiety.

“2′-substituted nucleoside” means a nucleoside comprising a substituent at the 2′-position of the furanosyl ring other than H or OH. In certain embodiments, 2′ substituted nucleosides include nucleosides with bicyclic sugar modifications.

“5-methylcytosine” means a cytosine modified with a methyl group attached to the 5 position. A 5-methylcytosine is a modified nucleobase.

“About” means within ±10% of a value. For example, if it is stated, “the compounds affected at least about 70% inhibition”, it is implied that levels are inhibited within a range of 60% and 80%.

“Administration” or “administering” refers to routes of introducing an antisense compound provided herein to a subject to perform its intended function. An example of a route of administration that can be used includes but is not limited to intravitreal administration. The intravitreal administration may be by direct injection, which means that the compound is injected straight into the vitreous of the subject's eye. The compound itself may be “naked”, or “as such”, but it may also be held in a delivery vehicle. When it is naked, it is generally contained in a formulation that besides the compound also comprises suitable and allowable pharmaceutical carriers, that are well-known to the person skilled in the art. Another preferred route of administration is by topical application of the pharmaceutical composition according to the present invention. It is preferred that the pharmaceutical composition, when administered topically, comprises a viscosifying polymer, preferably a non-ionic viscosifying polymer, more preferably hypromellose (HPMC).

“Amelioration” refers to a lessening of at least one indicator, sign, or symptom of an associated disease, disorder, or condition. In certain embodiments, amelioration includes a delay or slowing in the progression of one or more indicators of corneal dystrophy. The severity of indicators may be determined by subjective or objective measures, which are known to those skilled in the art.

“Antisense activity” means any detectable or measurable activity attributable to the hybridization of an antisense compound to its target nucleic acid. In certain embodiments, antisense activity is a decrease in the amount or expression of TGFBI mRNA and/or TGFBIp.

“Antisense compound” means an oligomeric compound that is capable of undergoing hybridization to a target nucleic acid, preferably human TGFBI mRNA, or a part thereof, through hydrogen bonding. A preferred antisense compound according to the invention is a single stranded antisense oligonucleotide (AON), more preferably a gapmer. The term AON is understood to refer to a nucleotide sequence which is substantially complementary to, and hybridizes to, a (target) (pre-) mRNA molecule. The degree of complementarity (or substantial complementarity) of the antisense sequence is preferably such that a molecule comprising the antisense sequence can form a stable double stranded hybrid with the target nucleotide sequence in the target RNA molecule under physiological conditions. The terms ‘AON’, ‘antisense oligonucleotide’, ‘oligonucleotide’ and ‘oligo’ are used interchangeably herein and are understood to refer to an oligonucleotide comprising an antisense sequence in respect of the target sequence. The AON of the present invention are not double stranded and are therefore not siRNAs. The AON of the present invention is man-made, and is chemically synthesized, generally in a laboratory by solid-phase chemical synthesis, followed by purification. It is typically purified or isolated.

“Antisense inhibition” means reduction of TGFBI transcript and TGFBIp levels in the presence of an antisense compound complementary to (a part of) TGFBI mRNA compared to TGFBI transcript and TGFBIp levels in the absence of the antisense compound, or in the presence of a non-targeting control antisense compound.

“Antisense mechanism” are all those mechanisms involving hybridization of an antisense compound with TGFBI target nucleic acid, wherein the outcome or effect of the hybridization is either TGFBI transcript degradation resulting in a decrease of the TGFBIp activity that is executed in the absence of the antisense compound.

“bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a furanosyl. In certain such embodiments, the bridge connects the 2′-carbon and the 4′-carbon of the furanosyl.

“Cap structure” or “terminal cap moiety” means chemical modifications, which have been incorporated at either terminus of an antisense compound.

“cEt” or “constrained ethyl” means a bicyclic sugar moiety comprising a bridge connecting the 4′-carbon and the 2′-carbon, wherein the bridge has the formula: 4′-CH(CH₃)—O-2′. “Constrained ethyl nucleoside (also cEt nucleoside) means a nucleoside comprising a bicyclic sugar moiety comprising a 4′-CH(CH₃)—O-2′ bridge.

“Chimeric antisense compounds” means antisense compounds that have at least two chemically distinct regions, which means that one region is in some way chemically different than another region of the same antisense compound, whereas each region has a plurality of subunits, and wherein the number of subunits is one or more.

“Complementarity” means the capacity for pairing between nucleobases of a first nucleic acid and a second nucleic acid. The term includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%.

The verb “to comprise” and its conjugations is used in its non-limiting sense to mean that items following the word are included, but items not specifically mentioned are not excluded.

“Deoxyribonucleotide” means a nucleotide having a hydrogen at the 2′ position of the sugar portion of the nucleotide. Deoxyribonucelotides may be modified with any of a variety of substituents.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings”. A gapmer is preferably used to target (pre-) mRNA to downregulate expression of a protein from that (pre-) mRNA by causing the degradation of the mRNA once the gapmer has formed a double-stranded structure with that target RNA.

“Internucleoside or internucleosidic linkage” refers to the chemical bond between nucleosides.

“Linked deoxynucleoside” means a nucleic acid base (A, G, C, T, U) substituted by deoxyribose linked by a phosphate ester to form a nucleotide.

“Mismatch” or “non-complementary nucleobase” refers to a case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” means any nucleobase other than adenine, cytosine, guanine, thymine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase. A gapmer, without any modifications in the sugar moiety, nucleobase or linkage may also be considered a modified oligonucleotide as it consists of wing-gap-wing segments that are—on a nucleotide level—different from one another (e.g., RNA-DNA-RNA).

“Modified sugar” or “modified sugar moiety” means substitution and/or change from a natural sugar moiety.

“Modulating” refers to changing or adjusting a feature in a cell, tissue, organ, or organism. For example, modulating TGFBI transcripts can mean to decrease the level (the amount; or number of copies) of TGFBI mRNA, or to decrease/influence the functionality of TGFBI protein in a cell, tissue, organ, or organism.

“Natural sugar moiety” means a sugar moiety found in DNA (2′-H) or RNA (2′-OH).

“Naturally occurring internucleoside linkage” means a 3′ to 5′ phosphodiester linkage.

“Nucleoside” means a nucleobase linked to a sugar.

“Nucleotide” means a nucleoside having a phosphate group covalently linked to the sugar portion of the nucleoside.

A “penetration enhancer” that is used in a composition of the present invention is preferably a viscosifying polymer. More preferably, the penetration enhancer is a non-ionic viscosifying polymer. A particularly preferred non-ionic viscosifying polymer that is used in a composition of the present invention is hypromellose, short for hydroxypropyl methylcellulose (HPMC), a semisynthetic, inert, visoelastic polymer often used as eye drops, as well as an excipient and controlled-delivery component in oral medicaments.

“Pharmaceutical composition” means a mixture of substances suitable for administering to an individual. For example, a pharmaceutical composition may comprise one or more active pharmaceutical agents (such as an oligonucleotide) and a sterile aqueous solution.

“Phosphorothioate linkage” (often abbreviated to PS linkage) means a linkage between nucleosides where the phosphodiester bond is modified by replacing one of the non-bridging oxygen atoms with a sulfur atom. A phosphorothioate linkage is a modified internucleoside linkage.

“Ribonucleotide” means a nucleotide having a hydroxy at the 2′ position of the sugar portion of the nucleotide. Ribonucleotides may be modified with any of a variety of substituents.

Certain embodiments provide antisense oligonucleotides (AONs), such as gapmers, targeting TGFBI transcripts. Certain embodiments provide methods, compounds, and compositions for inhibiting TGFBIp expression. In one embodiment, the present invention relates to AONs that are derivatives of the AONs of the present invention (for instance those that comprise additional nucleotides on either end, or that are made shorter by removal of nucleotides on either end), if their functionality (reducing TGFBI mRNA expression) remains present and can be determined and reaches a significant lower level.

In another preferred embodiment, the AON of the present invention comprises at least one non-naturally occurring chemical modification. Preferably, the non-naturally occurring modification comprises a modification of at least one internucleoside linkage. Preferred internucleoside linkage modifications are those in which a non-bridging oxygen atom is substituted by a sulfur atom, a phosphonate, a phosphorothioate (PS), a phosphodiester, a phosphoromorpholidate, a phosphoropiperazidate, a phosphonoacetate, a methylphosphonate, and a phosphoroamidate. The linkage modification is preferably a PS. In an even more preferred embodiment, all internucleoside linkages are chemically modified by a non-naturally occurring modification, and in a most preferred embodiment, all internucleoside linkages within the AON of the present invention carry a PS modification. The Sp or Rp configuration of each of these phosphorothioate linkage modifications may be selected to increase the binding efficiency to its target sequence, as well as its stability in vivo.

In another preferred embodiment, the AON of the present invention comprises one or more sugar moieties that is mono- or di-substituted at the 2′, 3′ and/or 5′ position. 2′-OMe and 2′-MOE modifications may both be present in a single AON of the present invention. The activity of each type of modified AON can be easily determined by the skilled person based on the teaching provided herein. The skilled person knows that such may depend on the cell type that is used, the way of introducing an AON into a cell, cell cycle state, etc. and that for each setting such may be tested and adjusted, which is all within the capabilities of the person skilled in the art.

In another embodiment, the invention relates to an AON according to the invention, wherein the AON is chemically linked to one or more conjugates that enhance the activity, the cellular distribution, or cellular uptake of the AON. WO 93/07883 and WO2013/033230 provide suitable conjugates, which are hereby incorporated by reference. In one embodiment, the conjugate is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids) or combinations thereof.

In another embodiment, the invention relates to a pharmaceutical composition comprising an AON according to the invention, and a pharmaceutically acceptable carrier. As outlined above, when the pharmaceutical composition is for topical application, it preferably comprises a penetration enhancer, more preferably a non-ionic viscosifying polymer such as hypromellose. In one aspect, the invention relates to an AON according to the invention, or a pharmaceutical composition according to the invention for use as a medicament. The invention relates to an AON according to the invention for use in the treatment of a TGFBI-related corneal dystrophy.

In a preferred embodiment, the AON of the present invention is administered and delivered ‘as is’, also referred to as ‘naked’. Nevertheless, the art contains multiple ways of delivering AONs to cells, either in vitro, ex vivo or in vivo. Depending on the disease, disorder or infection that needs to be treated, or on the cell, tissue or part of the body that needs to be reached by the AON of the present invention, an administration route or delivery method may be selected. Examples for delivery when the AON is not delivered naked, are delivery agents or vehicles such as nanoparticles, like polymeric nanoparticles, liposomes, antibody-conjugated liposomes, cationic lipids, polymers, or cell-penetrating peptides. As outlined above, TGFBI-related corneal dystrophies are preferably treated by administering the AON of the present invention through the direct application onto the outer layers of the eye (topically), wherein the pharmaceutical composition comprises, next to the AON and a solvent, also a penetration enhancer as further outlined herein, and in WO 2021/018750.

In another embodiment, the invention relates to a method of reducing expression of at least TGFBI transcript in a cell, comprising the step of administering to the cell an AON or a pharmaceutical composition according to the invention; optionally further comprising the step of determining whether the decrease in TGFBI transcript or TGFBIp levels in the cell has occurred, using methods known to the person skilled in the art. One can also determine the decrease in expression using a functional assay, for instance by determining whether corneal deposits have diminished in the eye, or whether vision in general is improved (which van be determined in a wide variety of ways). In a preferred embodiment, the cell used in the method of the present invention is an in vivo cell, or when cultured, an in vitro or ex vivo human cell, more preferably a target in vivo cell that is a corneal cell, even more preferably a corneal cell that is affected by TGFBI-induced deposits (granules, blobs, etc.).

In another embodiment, the invention relates to a method of treating a human subject suffering from a (TGFBI-related) corneal dystrophy, comprising the step of administering to the human subject an AON or a pharmaceutical composition according to the invention. Such administration may be topical, for instance by smearing a composition of the present invention directly on the eye, or by intravitreal injection.

In one embodiment, the invention relates to a method of modulating the level, expression and/or functionality of TGFBI mRNA and/or TGFBIp in a target cell, comprising the step of administering to the cell an AON or a pharmaceutical composition according to the invention; and allowing the cell to breakdown the double-stranded AON-target nucleic acid complex that is formed after the AON has entered the target cell. Preferably, the cell is a human cell. More preferably, the method is for modulating the level, expression, and/or function of TGFBIp by causing the breakdown of the AON-TGFBI double stranded complex, and thereby preventing, or inhibiting, or ameliorating the corneal dystrophy. Hence, the present invention relates to a method of treating, preventing, or ameliorating TGFBI-related corneal dystrophies.

In yet another embodiment, the invention relates to a use of an AON or a pharmaceutical composition according to the invention in the manufacture of a medicament for the treatment, prevention, or amelioration of a TGFBI-related corneal dystrophy.

The skilled person knows that an oligonucleotide, such as a chimeric antisense compound according to the present invention, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine, and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one aspect, the nucleobase in an AON of the present invention is adenine, cytosine, guanine, thymine, or uracil. In another aspect, the nucleobase is a modified form of adenine, cytosine, guanine, or uracil. In another aspect, the modified nucleobase is hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and Inosine' refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like. The sugar moiety can be a pyranose or derivative thereof, or a deoxypyranose or derivative thereof, preferably ribose or derivative thereof, or deoxyribose or derivative thereof.

A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O, 4′-C-ethylene-bridged nucleic acid (Morita et al. 2001. Nucleic Acid Res Supplement No. 1:241-242).

Sometimes the terms adenosine and adenine, guanosine and guanine, cytosine and cytidine, uracil and uridine, thymine and thymidine, inosine, and hypoxanthine, are used interchangeably to refer to the corresponding nucleobase, nucleoside, or nucleotide. Sometimes the terms nucleobase, nucleoside and nucleotide are used interchangeably, unless the context clearly requires differently. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

In one aspect, an AON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/PS monomers. It is noted that DNA is considered as an RNA derivative in respect of 2′ substitution. An AON of the present invention comprises at least one 2′-substituted RNA monomer connected through or linked by a PS or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. In a preferred embodiment of this aspect is provided an AON according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, any other 2′-substituted monomer within the AON is a 2′-substituted RNA monomer, such as a 2′-OMe RNA monomer or a 2′-MOE RNA monomer, which may also appear within the AON in combination.

Throughout the application, a 2′-OMe monomer within an AON of the present invention may be replaced by a 2′-OMe PS RNA, a 2′-OMe phosphate RNA or a 2′-OMe phosphate/PS RNA. Throughout the application, a 2′-MOE monomer may be replaced by a 2′-MOE PS RNA, a 2′-MOE phosphate RNA or a 2′-MOE phosphate/PS RNA. Throughout the application, an oligonucleotide consisting of 2′-OMe RNA monomers linked by or connected through PS, phosphate or mixed phosphate/PS backbone linkages may be replaced by an oligonucleotide consisting of 2′-OMe PS RNA, 2′-OMe phosphate RNA or 2′-OMe phosphate/PS RNA. Throughout the application, an oligonucleotide consisting of 2′-MOE RNA monomers linked by or connected through PS, phosphate or mixed phosphate/PS backbone linkages may be replaced by an oligonucleotide consisting of 2′-MOE PS RNA, 2′-MOE phosphate RNA or 2′-MOE phosphate/PS RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC} (NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are PS, chirally pure PS, Rp PS, Sp PS, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, N3→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

The present invention also relates to a chirally enriched population of modified AONs according to the invention, wherein the population is enriched for modified AONs comprising at least one particular PS internucleoside linkage having a particular stereochemical configuration, preferably wherein the population is enriched for modified AONs comprising at least one particular PS internucleoside linkage having the Sp configuration, or wherein the population is enriched for modified AONs comprising at least one particular PS internucleoside linkage having the Rp configuration.

In a preferred embodiment, the nucleotide analogue or equivalent comprises a modified backbone, exemplified by morpholino backbones, carbamate backbones, siloxane backbones, sulfide, sulfoxide, and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

It is further preferred that the linkage between the residues in a backbone do not include a phosphorus atom, such as a linkage that is formed by short chain alkyl or cycloalkyl internucleoside linkages, mixed heteroatom, and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively (Egholm et al. (1993) Nature 365:566-568).

It is understood by a skilled person that it is not necessary for all positions in an AON to be modified uniformly. In addition, more than one of the analogues or equivalents may be incorporated in a single AON or even at a single position within an AON. In certain embodiments, an AON of the invention has at least two different types of analogues or equivalents. A preferred gapmer according to the invention comprises a 2′-O alkyl PS antisense oligonucleotide, such as 2′-OMe modified ribose (RNA), 2′-O-ethyl modified ribose, 2′-O-propyl modified ribose, and/or substituted derivatives of these modifications such as halogenated derivatives. An effective AON according to the invention comprises a 2′-OMe ribose and/or a 2′-MOE ribose with a (preferably full) PS backbone.

It will also be understood by a skilled person that different AONs can be combined for efficiently downregulating the expression of TGFBI transcript. In a preferred embodiment, a combination of at least two AONs are used in a method of the invention, such as 2, 3, 4, or 5 different AONs. Hence, the invention also relates to a composition comprising a set of AONs comprising at least one AON according to the present invention, optionally further comprising AONs as disclosed herein.

An AON of the present invention can be linked to a moiety that enhances uptake of the AON in cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

Uridines in an AON according to the invention may be 5-methyluridine, or just uridine without a 5-methyl group in the base. Similarly, cytidines in an AON according to the invention may be 5-methylcytidine, or just cytidine without a 5-methyl group in the base. An AON according to the invention may contain one of more DNA residues, and/or one or more nucleotide analogues or equivalents, which means that a “U” as displayed in the sequences of the AONs may also be read as a “T” when it is DNA.

It is preferred that an AON of the invention comprises one or more residues that are modified to increase nuclease resistance when not attached to the target sequence, and/or to increase the affinity of the AON for the target sequence. Therefore, in a preferred embodiment, the AON sequence comprises at least one nucleotide analogue or equivalent, wherein a nucleotide analogue or equivalent is defined as a residue having a modified base, and/or a modified backbone, and/or a non-naturally occurring internucleoside linkage, or a combination of these modifications. Most preferably, all internucleoside linkages are modified, and all sugar moieties of the wing nucleosides are substituted at the 2′, 3′ and/or 5′ position, to render those parts of the oligonucleotide more resistant to breakdown.

The sugar moiety can be a pyranose or derivative thereof, or a deoxypyranose or derivative thereof, preferably ribose or derivative thereof, or deoxyribose or derivative thereof. A preferred derivatized sugar moiety, and non-naturally occurring chemical modification of the oligonucleotides of the present invention is Locked Nucleic Acid (LNA), in which the 2′-carbon atom is linked to the 3′ or 4′ carbon atom of the sugar ring thereby forming a bicyclic sugar moiety. As outlined in the accompanying examples, the introduction of several LNAs in the AONs of the present invention may increase the efficiency of skipping even further. A preferred LNA comprises 2′-O, 4′-C-ethylene-bridged nucleic acid. Other sugar modified nucleosides include, for example, bicyclohexose nucleic acids (WO2011/017521) or tricyclic nucleic acids (WO2013/154798).

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

In all embodiments of the invention, an AON is preferably a gapmer, more preferably a gapmer that reduces the expression of TGFBI transcript in corneal cells. A preferred AON of the present invention comprises or consists of a sequence selected from the group consisting of SEQ ID NO:1, 2, 3, 4, 5, 6, 7, 8, 9, 10, 11, 12, 13, 14, 15, 16, 19, 25, 26, 27, 30, 31, 33, 36, 37, 40, 41, 42, 43, 44, 46, 47, 48, 49, 50, 51, 52, 53, 54, 55, 56, 57, 58, 59, 60, 61, 62, 64, 65, 67, 68, 70, 71, 72, and 73, preferably from the group consisting of SEQ ID NO:1, 2, 3, 4, 5, 6, 7, 8, 9, 10, 11, 12, 13, 14, 15, 27, 33, 40, 42, 46, 47, 48, 49, 50, 51, 52, 53, 54, 55, 56, 57, 58, 59, 60, 62, 65, 70, 71, 72, and 73, and more preferably from the group consisting of SEQ ID NO:4, 12, and 13.

The invention provides a method for designing a gapmer able to downregulate expression of TGFBI transcript and/or TGFBIp in a corneal cell.

In a preferred method at least one of the following aspects must be considered for designing, improving said gapmers further: the AON preferably does not contain a CpG island or a stretch of CpG islands; and the AON has acceptable RNA binding kinetics and/or thermodynamic properties. The presence of a CpG or a stretch of CpG in an AON is usually associated with an increased immunogenicity of said AON. This increased immunogenicity is undesired since it may induce damage of the tissue to be treated. Immunogenicity may be assessed in an animal model by assessing the presence of CD4+ and/or CD8+ cells and/or inflammatory mononucleocyte infiltration. Immunogenicity may also be assessed in blood of an animal or of a human being treated with an AON of the invention by detecting the presence of a neutralizing antibody and/or an antibody recognizing said AON using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said AON using a standard immunoassay. The RNA binding kinetics and/or thermodynamic properties are at least in part determined by the melting temperature of an AON (Tm; calculated with an oligonucleotide properties calculator known to the person skilled in the art), and/or the free energy of the AON-target complex. If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol.

An AON of the invention is preferably one that can exhibit an acceptable level of functional activity. A functional activity of said AON is preferably to reduce the expression of TGFBI (pre-) mRNA to a certain acceptable level, to provide an individual with a non-toxic (or-disease causing) amount of TGFBIp. In a preferred embodiment, the decrease in expression is at least 2-10%, preferably at least 10-20%, more preferably at least 20-30%, even more preferably at least 30-40%, and most preferably at least 45%, at least 50%, at least 55%, at least 60%, at least 65%, at least 70%, at least 75%, at least 80%, at least 85%, at least 90%, at least 95%, or 100% as compared to a control RNA product not treated with an AON or a negative control AON. The present disclosure now enables the skilled person to generate a gapmer that provides significant decreased levels of TGFBI transcript in corneal cells.

In a preferred embodiment, the length of the complementary part for the AON of the invention is at least 10, 11, 12, 13, 14, 15, 16, 17, 18, 19, 20, 21, 22, 23, 24, 25, 26, 27, 28, 29 or 30 nucleotides. More preferably, the length of said complementarity is 14, 15, 16, 17, 18, 19, 20, 21, 22, 23, or 24 nucleotides. Most preferably, the length of said complementarity is 16 nucleotides, as exemplified herein. From an AON side, the preferred length of an AON according to the invention is at least 10, 11, 12, 13, 14, 15, 16, 17, 18, 19, 20, 21, 22, 23, 24, 25, 26, 27, 28, 29 or 30 nucleotides. Preferably, the length of an AON according to the invention is 14, 15, 16, 17, 18, 19, 20, 21, 22, 23, or 24 nucleotides. Most preferably, the length of a gapmer according to the invention is 16 nucleotides. Additional flanking sequences may be used to modify the binding of a protein to the AON, or to modify a thermodynamic property of the AON, more preferably to modify target RNA binding affinity. It is thus not absolutely required that all the bases in the region of complementarity are capable of pairing with bases in the opposing strand. For instance, when designing the AON, one may want to incorporate for instance a residue that does not base pair with the base on the complementary strand. Mismatching may, to some extent, be allowed, if under the circumstances in the cell, the stretch of nucleotides is sufficiently capable of hybridizing to the complementary part. In this context, ‘sufficiently’ preferably means that in a gel mobility shift assay as noted above, binding of an AON is detectable. The risk that the AON also will be able to hybridize to one or more other pre-mRNA molecules decreases with increasing size of the AON. It is clear that AONs that mismatch in the region of complementarity but that retain the capacity to hybridize and/or bind to the targeted region(s) in the pre-mRNA, can be used in the invention. However, preferably at least the complementary parts do not mismatch as AONs that do not mismatch in the complementary part typically have a higher efficiency and a higher specificity than AONs that do mismatch in one or more complementary regions. It is thought that higher hybridization strengths (i.e., increasing number of interactions with the opposing strand) are favorable in increasing the efficiency of the process of interfering with the splicing machinery of the system. An AON of the invention, when manufactured, is preferably an isolated single stranded antisense molecule in the absence of its (target) counterpart sequence.

It will also be understood by a skilled person that different gapmers can be combined for efficiently reducing TGFBIp expression in a corneal cell, preferably in the treatment of corneal dystrophy. In a preferred embodiment, a combination of at least two gapmers are used in a method of the invention, such as 2, 3, 4, or 5 different gapmers, more preferably wherein GM2 and GM3 are combined. Hence, the invention also relates to a set of gapmers comprising at least one gapmer according to the present invention. Nevertheless, from a regulatory and ease-of-production point of view, it is preferred that the medicament only comprises a single gapmer of the present invention.

An AON according to the invention can be delivered as is (i.e., naked and/or in isolated form) to an individual, an organ (the eye), or specifically to a corneal cell. When administering an AON according to the invention, it is preferred that the AON is dissolved in a solution that is compatible with the delivery method. Such delivery may be in vivo, in vitro or ex vivo. Nanoparticles and micro particles that may be used for in vivo AON delivery are well known in the art. Alternatively, a plasmid can be provided by transfection using known transfection reagents. For intravitreal administration it is preferred that the solution is a physiological salt solution. Particularly preferred in the invention is the use of an excipient or transfection reagents that will aid in delivery of each of the constituents as defined herein to a cell and/or into a cell (preferably a corneal cell affected by TGFBI-induced deposits, such as further outlined herein). Preferred are excipients or transfection reagents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), lipofectin™, DOTAP and/or viral capsid proteins that are capable of self-assembly into particles that can deliver each constituent as defined herein to a cell, preferably a corneal cell. Such excipients have been shown to efficiently deliver an AON to a wide variety of cultured cells. Their high transfection potential is combined with an excepted low to moderate toxicity in terms of overall cell survival. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidyl ethanolamine (DOPE). The neutral component mediates the intracellular release. Another group of delivery system are polymeric nanoparticles. Polycations such as diethylamino ethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver AONs across cell membranes into cells. In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an AON. The skilled person may select and adapt any of the above or other commercially available alternative excipients and delivery systems to package and deliver an AON for use in the current invention to deliver it for the treatment of corneal dystrophy.

An AON according to the invention could be covalently or non-covalently linked to a targeting ligand specifically designed to facilitate the uptake into the cell, cytoplasm, and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake into cells and/or the intracellular release of an oligonucleotide from vesicles, e.g., endosomes or lysosomes. Therefore, in a preferred embodiment, an AON according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an AON according to the invention and a further adjunct compound. If required, an AON according to the invention can be incorporated into a pharmaceutically active mixture by adding a pharmaceutically acceptable carrier. Accordingly, the invention also provides a composition, preferably a pharmaceutical composition, comprising an AON according to the invention and a pharmaceutically acceptable excipient. Such composition may comprise a single AON but may also comprise multiple, distinct AONs according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington (Remington. 2000. The Science and Practice of Pharmacy, 20th Edition. Baltimore, MD: Lippincott Williams Wilkins). Each feature of said composition has earlier been defined herein.

### EXAMPLES

### Example 1. Downregulation of TGFBI Transcript in Human A549 Cells After Transfection of Modified Gapmer Gapmers

The inventors designed a gapmer walk procedure to find a gapmer sequence potent enough to downregulate the TGFBI transcript and thereby lower the expression of TGFBIp. The gapmers covered the TGFBI transcript from exon 2 to exon 8 represented by SEQ ID NO:75 (see below). Gapmers comprising three or more consecutive G and/or C nucleotides were omitted. Initially, a set of 73 gapmers were designed and generated as LNA/DNA oligonucleotides with phosphorothioate (PS) backbone modifications (Table I). Each wing segment (both at the 3′ and at the 5′ end) consisted of three LNA nucleotides, whereas the gap segment consisted of 10 nucleotides that were all DNA. These 73 gapmers are represented by SEQ ID NO:1 to 73, respectively. As a negative control, a gapmer was taken along that does not hybridize to the TGFBI transcript. This control gapmer has the following sequence: 5′-AAC ACG TCT ATA CGC-3′ (SEQ ID NO:74).

(most of exon 2 to most
of exon 8 of human TGFBI);
SEQ ID NO: 75
5′-CAC UAA UAG GAA GUA CUU CAC CAA CUG
CAA GCA GUG GUA CCA AAG GAA AAU CUG UGG
CAA AUC AAC AGU CAU CAG CUA CGA GUG CUG
UCC UGG AUA UGA AAA GGU CCC UGG GGA
GAA GGG CUG UCC AGC AGC CCU ACC ACU CUC
AAA CCU UUA CGA GAC CCU GGG AGU CGU
UGG AUC CAC CAC CAC UCA GCU GUA CAC GGA
CCG CAC GGA GAA GCU GAG GCC UGA GAU
GGA GGG GCC CGG CAG CUU CAC CAU CUU CGC
CCC UAG CAA CGAG GCC UGG GCC UCC UUG
CCA GCU GAA GUG CUG GAC UCC CUG GUC AGC
AAU GUC AAC AUU GAG CUG CUC AAU GCC
CUC CGC UAC CAU AUG GUG GGC AGG CGA GUC
CUG ACU GAU GAG CUG AAA CAC GGC AUG
ACC CUC ACC UCU AUG UAC CAG AAU UCC AAC
AUC CAG AUC CAC CAC UAUC CUA AUG GGA
UUG UAA CUG UGA ACU GUG CCC GGC UGC UGA
AAG CCG ACC ACC AUG CAA CCA ACG GGG
UGG UGC ACC UCA UCG AUA AGG UCA UCU CCA
CCA UCA CCA ACA ACA UCC AGC AGA UCA
UUG AGA UCG AGG ACA CCU UUG AGA CCC UUC
GGG CUG CUG UGG CUG CAU CAG GGC UCA
ACA CGA UGC UUG AAG GUA ACG GCC AGU ACA
CGC UUU UGG CCC CGA CCA AUG AGG CCU
UCG AGA AGA UCC CUA GUG AGA CUU UGA ACC
GUA UCC UGG GCG ACC CAG AAG CCC UGA
GAG ACC UGC UGA ACA ACC ACA UCU UGA AGU
CAG CUA UGU GUG CUG AAG CCA UCG UUG
CGG GGC UGU CUG UAG AGA CCC UGG AGG GCA
CGA CAC UGG AGG UGG GCU GCA GCG GGG
ACA UGC UCA CUA UCA ACG GGA AGG CGA UCA
UCU CCA AUA AAG ACA UCC UAG CCA CCA
ACG GGG UGA UCC ACU ACA UUG AUG AGC UAC
UCA UCC CAG ACU CAG CCA AGA CAC UAU
UUG AAU UGG CU-3′

TABLE I
Sequences of gapmers

| | |
|---|---|
| Nr1 | 5′-GUA CTT CCT ATT AGU G-3′ |
| Nr2 | 5′-GUG AAG TAC TTC CUA U-3′ |
| Nr3 | 5′-AGU TGG TGA AGT ACU U-3′ |
| Nr4 | 5′-CUU GCA GTT GGT GAA G-3′ (GM1) |
| Nr5 | 5′-CAC TGC TTG CAG TUG G-3′ |
| Nr6 | 5′-GGU ACC ACT GCT TGC A-3′ |
| Nr7 | 5′-CCU TTG GTA CCA CUG C-3′ |
| Nr8 | 5′-AUU TTC CTT TGG TAC C-3′ |
| Nr9 | 5′-CAC AGA TTT TCC TUU G-3′ |
| Nr10 | 5′-AUG ACT GTT GAT TUG C-3′ |
| Nr11 | 5′-CUG ATG ACT GTT GAU U-3′ |
| Nr12 | 5′-UAG CTG ATG ACT GUU G-3′ (GM2) |
| Nr13 | 5′-UCG TAG CTG ATG ACU G-3′ (GM3) |
| Nr14 | 5′-CAC TCG TAG CTG AUG A-3′ |
| Nr15 | 5′-CAG CAC TCG TAG CUG A-3′ |
| Nr16 | 5′-GGA CAG CAC TCG TAG C-3′ |
| Nr17 | 5′-CCA GGA CAG CAC TCG U-3′ |
| Nr18 | 5′-UAU CCA GGA CAG CAC U-3′ |
| Nr19 | 5′-UUU CAT ATC CAG GAC A-3′ |
| Nr20 | 5′-GAC CTT TTC ATA TCC A-3′ |
| Nr21 | 5′-UAG TGG TGG ATC TGG A-3′ |
| Nr22 | 5′-UAG GAT AGT GGT GGA U-3′ |
| Nr23 | 5′-GUU CAC AGT TAC AAU C-3′ |
| Nr24 | 5′-GCA CAG TTC ACA GUU A-3′ |
| Nr25 | 5′-UGG TTG CAT GGT GGU C-3′ |
| Nr26 | 5′-CGU TGG TTG CAT GGU G-3′ |
| Nr27 | 5′-AUC GAT GAG GTG CAC C-3′ |
| Nr28 | 5′-ACC TTA TCG ATG AGG U-3′ |
| Nr29 | 5′-AGA TGA CCT TAT CGA U-3′ |
| Nr30 | 5′-GGU GGA GAT GAC CUU A-3′ |
| Nr31 | 5′-GUG ATG GTG GAG AUG A-3′ |
| Nr32 | 5′-UGU TGG TGA TGG TGG A-3′ |
| Nr33 | 5′-GAU GTT GTT GGT GAU G-3′ |
| Nr34 | 5′-UGC TGG ATG TTG TUG G-3′ |
| Nr35 | 5′-UGA TCT GCT GGA TGU U-3′ |
| Nr36 | 5′-CUC AAT GAT CTG CUG G-3′ |
| Nr37 | 5′-CGA TCT CAA TGA TCU G-3′ |
| Nr38 | 5′-GUC CTC GAT CTC AAU G-3′ |
| Nr39 | 5′-AAG GTG TCC TCG AUC U-3′ |
| Nr40 | 5′-UCU CAA AGG TGT CCU C-3′ |
| Nr41 | 5′-UCA AGC ATC GTG TUG A-3′ |
| Nr42 | 5′-CCU TCA AGC ATC GUG U-3′ |
| Nr43 | 5′-UUA CCT TCA AGC AUC G-3′ |
| Nr44 | 5′-GGU TCA AAG TCT CAC U-3′ |
| Nr45 | 5′-UUC AGC AGG TCT CUC A-3′ |
| Nr46 | 5′-GGU TGT TCA GCA GGU C-3′ |
| Nr47 | 5′-GAU GTG GTT GTT CAG C-3′ |
| Nr48 | 5′-CAA GAT GTG GTT GUU C-3′ |
| Nr49 | 5′-CUU CAA GAT GTG GUU G-3′ |
| Nr50 | 5′-UGA CTT CAA GAT GUG G-3′ |
| Nr51 | 5′-AGC TGA CTT CAA GAU G-3′ |
| Nr52 | 5′-CAU AGC TGA CTT CAA G-3′ |
| Nr53 | 5′-ACA CAT AGC TGA CUU C-3′ |
| Nr54 | 5′-AGC ACA CAT AGC TGA C-3′ |
| Nr55 | 5′-UUC AGC ACA CAT AGC U-3′ |
| Nr56 | 5′-GAU GGC TTC AGC ACA C-3′ |
| Nr57 | 5′-UGA TAG TGA GCA TGU C-3′ |
| Nr58 | 5′-UUA TTG GAG ATG AUC G-3′ |
| Nr59 | 5′-UGU CTT TAT TGG AGA U-3′ |
| Nr60 | 5′-GGA TGT CTT TAT TGG A-3′ |
| Nr61 | 5′-CUA GGA TGT CTT TAU U-3′ |
| Nr62 | 5′-AUC AAT GTA GTG GAU C-3′ |
| Nr63 | 5′-AGC TCA TCA ATG TAG U-3′ |
| Nr64 | 5′-UGA GTA GCT CAT CAA U-3′ |
| Nr65 | 5′-AUU CAA ATA GTG TCU U-3′ |
| Nr66 | 5′-CUG CAG CAC CAG CUG G-3′ |
| Nr67 | 5′-CCU TCA GGA CAT CCA U-3′ |
| Nr68 | 5′-AGG ATT TCA TCA CCA A-3′ |
| Nr69 | 5′-UAA CCA GGA TTT CAU C-3′ |
| Nr70 | 5′-AAU GCT TCA TCC TCU C-3′ |
| Nr71 | 5′-CAA CTC ATA GCT TAU A-3′ |
| Nr72 | 5′-GUU TTA TTA TTA CAA A-3′ |
| Nr73 | 5′-CUU TGG TTT TAT TAU U-3′ |

In a first experiment, all 73 gapmers were assayed in human A549 cells that express endogenous TGFBIp. Cells were grown in 12-well plate format culture plate with RPMI 1640 and 10% FBS and seeded in a density of 1×10⁵ cells per well after which they were cultured for 24 h at 37° C. and 5% CO₂. Then, 100 nM of each gapmer was introduced into the cells in a biological duplicate by Lipofectamine-mediated transfection (Lipofectamine 2000, Invitrogen) at 1:1 mass transfection ratio. A gapmer mixture was created by adding 22 μl of a 10 μM gapmer solution to 88 μl PBS pH7.4 (Gibco). A Lipofectamine 2000 mixture was created by adding the same amount (μg of gapmer in the gapmer mixture) of Lipofectamine 2000 (Lipofectamine 2000, Invitrogen) to PBS pH7.4 (Gibco) in a total volume of 100 μl. Both mixtures were left at RT for 5 min before bringing them together. The combined gapmer-Lipofectamine mixture was left at RT for 20 min and then added to the cells together with 900 μl RPMI fresh medium. As a further control, a non-treated sample was included. Cells in this sample were not dosed with a gapmer and no Lipofectamine was added to the culture medium. After 24 h cells were rinsed with PBS (Gibco) and harvested in 350 μl Buffer RLT (RNeasy Plus Mini Kit, Qiagen) per well and RNA was isolated according to the manufacturer protocol (RNeasy Plus Mini Kit, Qiagen). RNA was eluted in 20 μl RNase free water (RNeasy Plus Mini Kit, Qiagen). RNA concentration was determined using NanoDrop (ThermoFisher Scientific) and stored at −80° C. until further use.

For cDNA synthesis 250 ng of total RNA was used according to the manufacturer recommendations (Maxima Reverse Transcriptase, ThermoFisher). In a negative control cDNA reaction, nuclease-free water was used instead of Maxima Reverse Transcriptase (ThermoFisher). cDNA was synthesized in a T100 Thermal Cycler (BioRad) and stored at −20° C.

TGFBI transcripts were detected in a technical duplicate using a TaqMan assay (Hs00932747_m1, Applied Biosystems (FAM)) in a digital droplet PCR (ddPCR), which was performed using 4 μl of a 32× diluted cDNA solution per reaction. To normalize TGFBI transcripts,

GUSB transcripts were analysed in a technical duplicate. Reaction mixtures were made with 5.25 μl Supermix ddPCR Supermix for Probes (no dUTP) (BioRad), 0.525 μl Taqman assay and 2.725 μl nuclease free water. A ddPCR was performed by adding 2 μl of an 8× diluted cDNA solution to the reaction mixture per reaction. Transcript levels (total copies) were measured according to the manufacturer recommendations. The QX200 Droplet generator was used to generate droplets and PCR was run on a T100 Thermo Cycler (BioRad). Upon PCR, droplets were analysed on the QX200 droplet reader (BioRad) and data analysis was performed using the Quantasoft software (BioRad). Only samples in which more than 8000 droplets were detected were included in the data analysis. Water and minus RT samples were included as negative controls. TGFBI transcript levels were normalized to GUSB transcript levels by calculating the TGFBI/GUSB ratio for each sample using the average droplet vales of the technical duplicates. Normalized TGFBI expression (TGFBI/GUSB ratio) in gapmer-treated samples was compared to the normalized TGFBI expression of the non-treated sample to determine knockdown levels by dividing the TGFBI/GUSB ratio of a gapmer treated sample by the TGFBI/GUSB ratio of the non-treated sample. The percentages knockdown achieved with the 73 gapmers are shown in Table II.

From Table II it was concluded that some gapmers work better than others, which is not unexpected. Good performing and preferred gapmers are Nr1, 2, 3, 4, 5, 6, 7, 8, 9, 10, 11, 12, 13, 14, 15, 16, 19, 25, 26, 27, 30, 31, 33, 36, 37, 40, 41, 42, 43, 44, 46, 47, 48, 49, 50, 51, 52, 53, 54, 55, 56, 57, 58, 59, 60, 61, 62, 64, 65, 67, 68, 70, 71, 72, and 73. Specifically preferred are Nr1, 2, 3, 4, 5, 6, 7, 8, 9, 10, 11, 12, 13, 14, 15, 27, 33, 40, 42, 46, 47, 48, 49, 50, 51, 52, 53, 54, 55, 56, 57, 58, 59, 60, 62, 65, 70, 71, 72, and 73 that were all performing with efficiencies over 10%. Three particularly well performing gapmers were identified: gapmer Nr4 (SEQ ID NO:4), also herein referred to as GM1, showed 80% knockdown of the TGFBI transcript; gapmer Nr12 (SEQ ID NO:12), also herein referred to as GM2, reduced TGFBI expression with 81%; and gapmer Nr13 (SEQ ID NO:13), also herein referred to as GM3 showed a knockdown efficiency of 78%. In conclusion, the inventors identified new and very efficient human TGFBI-targeting gapmers that can downregulate the expression of the human TGFBI transcript in a target cell, here exemplified by human A549 cells.

TABLE II
Relative TGFBI mRNA knockdown percentage (normalized to a non-
transfected sample) in A549 cells upon Lipofectamine 2000 transfection
of 100 nM gapmer. Positive percentages show knockdown; 0% and
lower did not show knockdown (in this experiment).

| | | |
|---|---|---|
| Nr1 | 79 | % |
| Nr2 | 69 | % |
| Nr3 | 69 | % |
| Nr4 | 80 | % |
| Nr5 | 67 | % |
| Nr6 | 72 | % |
| Nr7 | 29 | % |
| Nr8 | 74 | % |
| Nr9 | 48 | % |
| Nr10 | 69 | % |
| Nr11 | 57 | % |
| Nr12 | 81 | % |
| Nr13 | 78 | % |
| Nr14 | 53 | % |
| Nr15 | 66 | % |
| Nr16 | 2 | % |
| Nr17 | −3 | % |
| Nr18 | −1 | % |
| Nr19 | 10 | % |
| Nr20 | 0 | % |
| Nr21 | −4 | % |
| Nr22 | −3 | % |
| Nr23 | −10 | % |
| Nr24 | 0 | % |
| Nr25 | 3 | % |
| Nr26 | 8 | % |
| Nr27 | 13 | % |
| Nr28 | −3 | % |
| Nr29 | −4 | % |
| Nr30 | 2 | % |
| Nr31 | 5 | % |
| Nr32 | −4 | % |
| Nr33 | 13 | % |
| Nr34 | −47 | % |
| Nr35 | −2 | % |
| Nr36 | 8 | % |
| Nr37 | 5 | % |
| Nr38 | −6 | % |
| Nr39 | −24 | % |
| Nr40 | 13 | % |
| Nr41 | 8 | % |
| Nr42 | 11 | % |
| Nr43 | 9 | % |
| Nr44 | 4 | % |
| Nr45 | −11 | % |
| Nr46 | 14 | % |
| Nr47 | 31 | % |
| Nr48 | 22 | % |
| Nr49 | 42 | % |
| Nr50 | 47 | % |
| Nr51 | 39 | % |
| Nr52 | 25 | % |
| Nr53 | 14 | % |
| Nr54 | 24 | % |
| Nr55 | 30 | % |
| Nr56 | 16 | % |
| Nr57 | 35 | % |
| Nr58 | 38 | % |
| Nr59 | 28 | % |
| Nr60 | 16 | % |
| Nr61 | 2 | % |
| Nr62 | 16 | % |
| Nr63 | 0 | % |
| Nr64 | 6 | % |
| Nr65 | 16 | % |
| Nr66 | 0 | % |
| Nr67 | 10 | % |
| Nr68 | 6 | % |
| Nr69 | 0 | % |
| Nr70 | 18 | % |
| Nr71 | 16 | % |
| Nr72 | 15 | % |
| Nr73 | 12 | % |

GM1, GM2, and GM3 were analyzed in eight individual experiments to further assess TGFBI knockdown efficiency. In each experiment, A549 cells were grown as indicated above and 100 nM gapmer was used in a transfection experiment as outlined above. RNA was isolated and cDNA was synthesized as described. TGFBI transcripts were detected in a technical triplicate using a TaqMan assay in a quantitative real time PCR (qPCR) assay, that was performed using 4 μl of an 8× diluted cDNA solution per reaction. To normalize TGFBI transcripts, GUSB transcripts were analysed in a technical triplicate by qPCR using a TaqMan assay (Applied Biosystems). Reaction mixtures were made with 10 μl TaqMan™ Fast Advanced Master Mix (ThermoFisher Scientific), 1 μl of TGFBI or GUSB FAM labelled probe and 5 μl nuclease-free water. qPCR was run on a 7900HT Fast Real-Time PCR system (Applied Biosystems). Ct values were measured by CFX Maestro software (BioRad). As negative controls, a reaction without cDNA template and a reaction with cDNA created with nuclease free water instead of Maxima Reverse were included. Ct values were used to calculate the dCt values (Ct_{TGFBI}-Ct_{GUSB}) of each technical replicate. The average dCt values of the technical triplicate was used to calculate the ddCt value (dCtₛₐₘₚₗₑ-dCt_{avarage non treated sample}) for each biological replicate. In this calculation dCt_{avarage non treated sample} was calculated as the average dCt value of both biological samples. Relative TGFBI expression in gapmer treated samples compared to the non-treated sample was determined by calculating the 2\hat{ }-ddCt values. The average relative TGFBI expression of the two biological replicates was determined for each individual experiment. A Kruskal-Wallis test was performed on relative TGFBI expression because the data points within the different treatment groups (non-treated (NT), negative control gapmer (Ctr(−)), GM1, GM2 and GM3 treated) did not pass the Anderson-Darling, Shapiro-Wilk and Kolmogorov-Smirnov normality test.

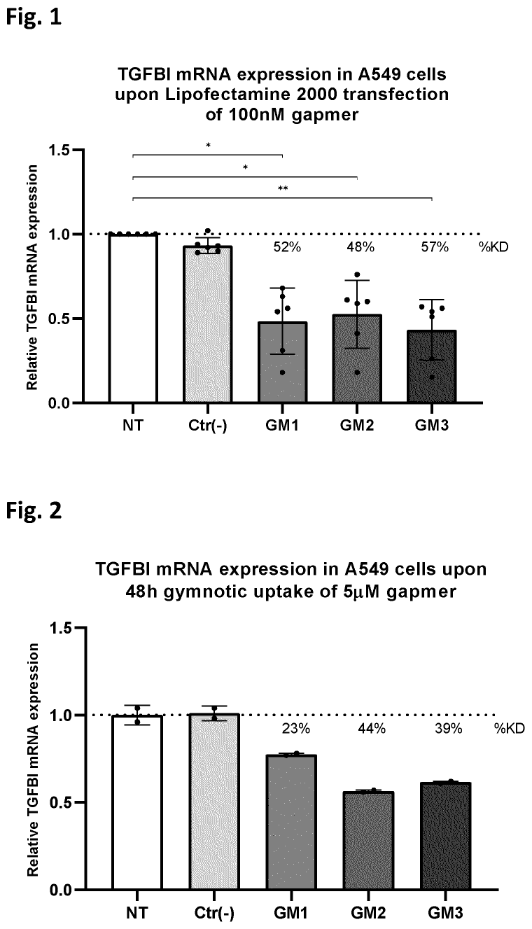

The results are shown in FIG. 1 and indicate a significant reduction in TGFBI expression with all three gapmers used. For GM1 a knock down efficiency of 52% (p=0.0105) was detected. GM2 showed a TGFBI expression reduction of 48% (p=0.0288). TGFBI transcript levels were reduced with 58% (p=0.0012) in GM3 treated samples, confirming the results found with the ddPCR.

### Example 2. Downregulation of TGFBI Transcript in Human A549 Cells Using Gymnotic Uptake of Modified Gapmer Gapmers

The inventors then questioned whether it was possible to show downregulation of TGFBI transcript levels without using an assisted uptake method. GM1, GM2, and GM3 were presented ‘naked’ (without any transfection reagent) to human A549 cells to assess the functional effect after gymnotic uptake (gymnosis). Cells were cultured as described in the previous example. Each gapmer was added in a 5 μM final concentration (in 1 ml of RPMI 1640 culture medium) in a biological duplicate to the cells. Cells were then cultured for 48 h, rinsed with PBS, and harvested. RNA isolation and cDNA synthesis were performed as described above. A qPCR assay was also performed as described above.

The results are given in FIG. 2 and show a 23% reduction in TGFBI expression for GM1 treated samples. For the GM2 treated sample a knock down efficiency of 44% was detected and for the GM3 treated sample this efficiency was 39%.

### Example 3. Downregulation of TGFBI Transcript and TGFBIp in Human A549 Cells Using Gymnotic Uptake of Modified Gapmers

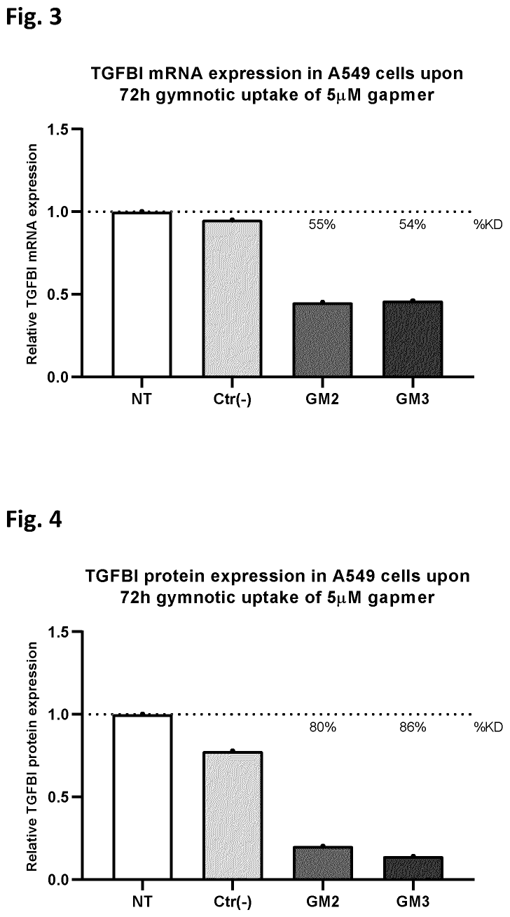

In a next experiment, GM2 and GM3 were introduced by gymnotic uptake as described above in human A549 cells and the downregulation of the protein encoded by TGFBI transcripts was assessed. Each gapmer was added in a 5 μM final concentration (in 1 ml of RPMI 1640 culture medium) in 12 wells and cells were cultured for 72 h. RNA isolation and cDNA synthesis were performed as described above. TGFBI transcripts were also detected as described above in a qPCR assay. FIG. 3 shows that TGFBI transcript levels were decreased with 55% after GM2 uptake and 54% after GM3 uptake.

For protein analysis, cells from eleven wells (from a 12-well plate) were rinsed with 500 μl PBS per well. Cells were harvested in 40 μl RIPA buffer (Sigma-Aldrick) containing protease inhibitor cocktail (Complete Tablets Mini EDTA-free EASY pack, Roche) per well. The monolayer of cells was scraped using a pipet tip, while the plate was placed on ice. Lysed cells were immediately transferred from all wells, pulled to a pre-cooled 1.5 ml Eppendorf tube and homogenized for 40 min at 4° C. Lysates were centrifuged at 14,000 rpm for 20 min at 4° C. and stored at −20° C. until further use.

To determine protein concentration a Bradford assay (Pierce™ BCA Protein Assay Kit, Thermo Scientific) was performed according to the manufacturer recommendations. Cell lysates were heated for 10 min at 95° C., vortexed, spun down shortly and kept on ice. A 10% Mini-Protean TGX stain-free precast gel with 50 μl wells (BioRad) was placed in a gel cassette according to the manufacturer protocol. Running buffer was made by diluting 100 ml 10× Tris-Glycine-SDS (BioRad) in 900 ml demi-water. Running buffer was added to the tank. 30 μg cell lysate sample was denatured with 4× Laemli buffer (BioRad) and 1M DTT (Sigma-Adrich). The volumes of 4× Laemli buffer and DTT used were calculated as a 33% and 40% of protein extract volume, respectively. Protein samples were loaded on the precast gel. ProSieve™ QuadColor™ protein marker, 4.6 kDa-300 kDa (Lonza) was loaded as well to control for protein seize. Gel was run and then placed in a Trans-Blot Turbo Transfer System/Membrane Mini format (BioRad) using a Transblot Turbo Membrane (BioRad) according to manufacturer instructions. Protein was transferred to membrane and the membrane was incubated for 1 h in Intercept Blocking Buffer (LI-COR) on a roller bench at RT using 5 ml blocking buffer. The membrane was washed once with 5 mL PBST (PBS/0.05% Tween 20). The membrane was incubated with primary antibody (1:1000 antibody solution prepared in 5 ml PBST) on a roller bench overnight at 4° C. As primary antibody, TGFBI Recombinant Rabbit Monoclonal Antibody (JM24-53; Invitrogen) was used. The membrane was washed three times for 5 min with 5 ml PBST the next day and incubated for 1 h at RT in the dark with IRDye® 800CW Goat anti-Rabbit IgG Secondary Antibody (LI-COR) (1:5000 antibody solution prepared in 5 ml PBST). The membrane was washed 3× for 5 min with 5 ml PBST and once with 5 ml PBS. As a loading control, the membrane was incubated with 1:1000 primary antibody in 5 ml PBST on a roller bench for 1 h at RT. As primary antibody, Anti-beta-Actin Antibody, clone RM112 (Milipore) was used. The membrane was washed 3× for 5 min with 5 ml PBST and incubated for 1 h at RT in the dark with IRDye® 680RD Goat anti-Rabbit IgG Secondary Antibody (LI-COR) prepared at 1:5000 in 5 ml PBST. The membrane was washed 3× for 5 min with 5 ml PBST and then once with 5 ml PBS. The membrane was scanned in the Odyssey CLx Imaging System (Oddysey) using both 700 and 800 nm infrared wavelength channels and the automatic imaging settings (84 μm, medium quality). Using Image Studio for Odyssey CLx software, TGFBIp and beta-Actin protein-specific bands (75 and 42 kDa, respectively) were detected and the intensity of signal per band (pixel density) was measured. TGFBIp signals were normalized to beta-Actin protein signal by calculating the TGFBIp/beta-Actin ratio for each sample. The normalized TGFBIp signal of a gapmer-treated sample was divided by the normalized TGFBIp signal of the non-treated sample to determine the relative TGFBIp expression and knockdown efficiency.

The results are given in FIG. 4. These show that TGFBIp levels decreased in gapmer-treated samples. In the GM2-treated sample a TGFBIp knockdown efficiency of no less than 80% was detected. For the GM3-treated sample TGFBIp expression decreased even by 86%. This shows that the inventors were not only able to obtain functional effect of downregulation in TGFBI transcript expression, but also in TGFBIp expression in cells after gymnotic uptake of a chemically modified gapmer targeting the human TGFBI mRNA.

### Example 4. Downregulation of TGFBI Transcript in Primary Human Keratocytes

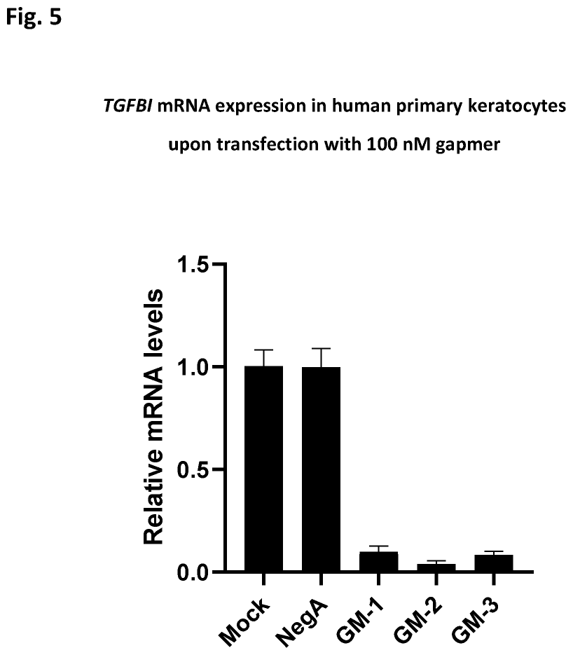

Next, it was investigated whether the gapmers GM1, GM2 and GM3 could downregulate expression of TGFBI mRNA in primary human keratocytes (HCK; Cell applications, California, USA). For this, cells were cultured in HCK Growth Medium (Cell applications) and passaged at 70-90% confluency every 3-4 days. All cells were maintained at 37° C. in a humified atmosphere of 5% CO₂. Approximately 150,000 cells per 12 well plate were seeded 16 hrs before transfection, which was performed with 100 nM gapmers and Lipofectamine 2000 Transfection Reagent (ThermoFisher) according to the manufacturer's instructions. Next to a mock transfection (no gapmer) a negative control gapmer with the sequence of SEQ ID NO:74 was taken along. All gapmers were individually mixed with Lipofectamine 2000 in a 1:2 ratio in Opti-MEM® I (1×) Reduced serum medium (Gibco; ThermoFisher) and incubated for 20 min at RT. RNA was extracted 24 hrs after transfection using the ReliaPrep™ RNA Miniprep System (Promega) according to manufacturer's protocol. cDNA was synthesized with an input of 250 ng RNA using the Maxima reverse transcription kit (ThermoFisher) according to the manufacturer's protocol in combination with oligo-dT primers and random hexamers. As a template 4 μL of 8 times diluted cDNA was used in combination with ready to use qPCR Taqman™ assays and TaqMan™ Fast Advanced Master Mix. The samples were analysed on a CFX96 thermal cycler (BioRad). The thermoprofile started with an initial activation at 50° C. for 2 min and then pre melting at 90° C. for 10 min. These steps were followed by 40 cycles of an annealing/extension phase based of 95° C. for 15 sec followed by 60° C. for 1 min. Cq values were estimated using the automatic threshold and baseline cycles option in the CFX Manager software. Relative expression was calculated using the delta ct analysis. Results shown in FIG. 5 indicate that treatment with all three selected TGFBI gapmers (GM1, GM2, and GM3) result in significant TGFBI mRNA reduction in comparison to the mock transfection (set at ratio 1.0) and the results obtained with the negative control gapmer, also in human primary keratocytes.

1. An antisense oligonucleotide (AON) capable of downregulating the expression of human Transforming Growth Factor Beta-Induced (TGFBI) transcript in a target cell, wherein the AON is 90% to 100% complementary to a consecutive stretch of nucleotides within the human TGFBI mRNA sequence of SEQ ID NO:75, wherein the AON consists of 16 to 30 linked nucleotides, and wherein the AON comprises:
(i) a gap segment consisting of at least ten deoxynucleotides;
(ii) a 5′ wing segment consisting of at least three nucleotides; and
(iii) a 3′ wing segment consisting of at least three nucleotides;
wherein the gap segment is positioned between the 5′ and 3′ wing segments, and wherein each wing segment comprises at least one nucleotide with a non-naturally occurring chemical modification in the sugar moiety.

2. The AON according to claim 1, wherein the AON comprises or consists of a sequence selected from the group consisting of SEQ ID NO:4, 12, 13, 1, 2, 3, 5, 6, 7, 8, 9, 10, 11, 14, 15, 16, 19, 25, 26, 27, 30, 31, 33, 36, 37, 40, 41, 42, 43, 44, 46, 47, 48, 49, 50, 51, 52, 53, 54, 55, 56, 57, 58, 59, 60, 61, 62, 64, 65, 67, 68, 70, 71, 72, and 73.

3. The AON according to claim 1, wherein the 5′ and/or the 3′ wing segment consists of LNA nucleotides.

4. The AON according to claim 1, wherein the 5′ and/or the 3′ wing segment comprises a nucleotide with a sugar moiety that is mono- or di-substituted at the 2′, 3′ and/or 5′ position, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O—, S- , or N-alkyl; —O—, S-, or N-alkenyl; —O—, S-, or N-alkynyl; —O—, S-, or N-allyl; —O-alkyl-O-alkyl; -methoxy; -aminopropoxy; -methoxyethoxy; -dimethylaminooxyethoxy; and -dimethylaminoethoxyethoxy.

5. The AON according to claim 1, wherein the AON comprises at least one phosphorothioate internucleoside linkage.

6. The AON according to claim 1, wherein the 3′ wing segment consists of three nucleotides, wherein the 5′ wing segment consists of three nucleotides and wherein the gap segment consists of ten nucleotides.

7. The AON according to claim 1, wherein the AON is for use in the prevention, treatment, or amelioration of a corneal dystrophy, preferably a corneal dystrophy caused by a mutated TGFBI gene, such as epithelial basement membrane dystrophy (EBMD), Reis Bucklers corneal dystrophy (RBCD), Thiel Behnke corneal dystrophy (TBCD), Lattice Corneal Dystrophy classic (LCD), Granular Corneal Dystrophy classic (GCD1), granular corneal dystrophy type II (GCD2), or Avellino Corneal Dystrophy (ACD).

8. A pharmaceutical composition comprising an AON according to claim 1, and a pharmaceutically acceptable carrier.

9. The pharmaceutical composition according to claim 8, further comprising a penetration enhancer.

10. The pharmaceutical composition according to claim 9, wherein the penetration enhancer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine.

11. (canceled)

12. (canceled)

13. The AON according to claim 5, wherein all of the internucleoside linkages are phosphorothioate internucleoside linkages.

14. A method for treating a patient suffering from a corneal dystrophy comprising administering to the patient a therapeutically effective amount of an AON according to claim 1.

15. The method according to claim 14, wherein the patient has a TGFBI gene mutation.

16. The method according to claim 14, wherein corneal dystrophy is selected from the group consisting of: epithelial basement membrane dystrophy (EBMD), Reis Bucklers corneal dystrophy (RBCD), Thiel Behnke corneal dystrophy (TBCD), Lattice Corneal Dystrophy classic (LCD), Granular Corneal Dystrophy classic (GCD1), granular corneal dystrophy type II (GCD2), or Avellino Corneal Dystrophy (ACD).

17. The method according to claim 15, wherein corneal dystrophy is selected from the group consisting of: epithelial basement membrane dystrophy (EBMD), Reis Bücklers corneal dystrophy (RBCD), Thiel Behnke corneal dystrophy (TBCD), Lattice Corneal Dystrophy classic (LCD), Granular Corneal Dystrophy classic (GCD1), granular corneal dystrophy type II (GCD2), or Avellino Corneal Dystrophy (ACD).

18. The method according to claim 14, wherein the AON is topically administered to the eye of the patient.
